(19) United States
(12) Patent Application Publication
(10) Pub. No.: US 2021/0287300 A1
(43) Pub. Date: Sep. 16, 2021

(54) INTELLIGENT IN-AND-OUT OF NETWORK SOLUTION (IIONS) SYSTEM

(71) Applicant: iiONS, LLC, Scottsdale, AZ (US)

(72) Inventor: Gregory M. Maldonado, Phoenix, AZ (US)

(21) Appl. No.: 17/200,567

(22) Filed: Mar. 12, 2021

Related U.S. Application Data
(60) Provisional application No. 62/988,560, filed on Mar. 12, 2020.

(51) Int. Cl. G06Q 40/08; G16H 80/00; G06F 40/289; G06F 40/126; G06F 40/174

(52) U.S. Cl. CPC G06Q 40/08; G16H 80/00; G06F 40/174; G06F 40/126; G06F 40/289

(57) ABSTRACT

Introduced here is an intelligent in-and-out of network solution (iiONS) system that has been developed to serve as an “all-in-one” dashboard for internal and external processing of claims. At a high level, the iiONS system can integrate with electronic medical record (EMR) systems to acquire, collect, or otherwise obtain data related to patients and claims. This data can be stored in central database tables maintained by the iiONS system. The central database tables may be reflected in user interfaces (or simply “interfaces”) that are generated by the iiONS system. These interfaces may be representative of a “portal” that can be used by analysts employed by a processing service for internal processing of claims.

### CROSS-REFERENCE TO RELATED APPLICATIONS

This application claims priority to U.S. Provisional Application No. 62/988,560, titled “Intelligent In-and-Out of Network Solution (iiONS) System” and filed on Mar. 12, 2020, which is incorporated by reference herein in its entirety.

### TECHNICAL FIELD

Various embodiments concern computer programs and associated computer-implemented techniques for facilitating the processing of claims in a semi- or fully-automated manner.

### BACKGROUND

Health insurance plans provide covered individuals (also referred to as “insureds” or patients”) access to a network of healthcare professionals and facilities. Collectively, these healthcare professionals and facilities may be referred to as “providers” of services that are covered by those health insurance plans. Examples of services include those billed for physicians, specialists, facilities (e.g., hospitals, clinics, and pharmacies), equipment (e.g., beds, ventilators, slings, and casts), radiology services, laboratory services, and pharmacology services.

Generally, providers must not only meet credentialing requirements, but also must agree to accept a discounted rate for covered services in order to be part of the network. Providers that accept those terms will be considered “in-network providers” by the insurer. If a provider has no contract with the insurer, however, then the provider will be considered an “out-of-network provider” by the insurer, and thus can charge patients the full price of any services provided.

### BRIEF DESCRIPTION OF THE DRAWINGS

FIG. 1 includes a high-level diagrammatic illustration of a process in which services provided by a healthcare professional at a partner facility are billed to an insurer by the iiONS system.

FIG. 2 includes a high-level diagrammatic illustration of another process in which services provided by a healthcare professional at a partner facility are billed to an insurer by the iiONS system.

FIG. 3 includes a high-level diagrammatic illustration of an external billing process facilitated by the iiONS system.

FIG. 4 includes a block diagram illustrating how the iiONS system capable of formulating requests for payment on behalf of healthcare professionals and partner facilities may be implemented.

FIG. 5 includes a block diagram illustrating the internal and external dataflows of the iiONS system in one embodiment.

FIGS. 6A-G include examples of interfaces that illustrate how a claim for a service provided to a patient can be generated.

FIGS. 7A-E include examples of interfaces that illustrate how tasks can be assigned, for example, to analysts employed by a processing service.

FIGS. 8A-B include examples of interfaces that illustrate how appeals can be initiated by the iiONS system.

FIG. 9 includes an example of an interface through which notes regarding suspension can be added.

FIG. 10 includes a flow diagram of a process for submitting a request for reimbursement to an insurer for a service provided to a patient.

FIG. 11 includes a flow diagram of a process for generating a claim on behalf of a healthcare professional and facility.

FIG. 12 includes a flow diagram of a process for creating a record for a patient to whom a service has been provided for which reimbursement will be sought.

FIG. 13 is a block diagram illustrating an example of a processing system in which at least some operations described herein can be implemented.

Various features of the technologies described herein will become more apparent to those skilled in the art from a study of the Detailed Description in conjunction with the drawings. Embodiments are illustrated by way of example and not limitation in the drawings. While the drawings depict various embodiments for the purpose of illustration, those skilled in the art will recognize that alternative embodiments may be employed without departing from the principles of the technologies. Accordingly, while specific embodiments are shown in the drawings, the technology is amenable to various modifications.

### DETAILED DESCRIPTION

For in-network providers covered by a health insurance plan, an insurer will agree to a set schedule of fees that are to be paid to those providers for services rendered. Notably, these fees are agreed to ahead of time, so while the insurer may not know which ailments might affect the patients covered by the health insurance plan, the insurer will know how much to pay for each service that is provided by one of the in-network providers. Normally, the insurer agrees to cover a percentage of the established billable fees under the agreement, and the insureds are responsible for covering a specific percentage of the remaining balance due to the providers in the form of copayments or deductibles.

In-network providers are normally sufficient for providing standard care and completing routine visits. However, there are many scenarios where coverage by in-network providers is impossible or impractical. Assume, for example, that a patient seeks treatment in an emergency room for a serious or complicated illness with sudden onset. That patient will most likely be treated by a team of healthcare professionals, some of whom may be considered in-network providers and some of whom may be considered out-of-network providers. Even if the health insurance plan covers services provided by the emergency room, some of the healthcare professionals working in that emergency room may not be included in the network, and therefore will not be subject to the set schedule of fees.

As with any contract, it is important for patients and providers to understand what services are covered by a health insurance plan before services are provided (and fees are incurred). This has historically been quite difficult, however. Introduced here, therefore, is an intelligent in-and-out of network solution (iiONS) system that has been developed to serve as an “all-in-one” dashboard for internal and external processing of claims. The term “internal processing” may refer to the conduct of a processing service that is responsible for processing claims, while the term “external processing” may refer to the conduct of providers—either in-network or out-of-network—that submit claims for processing.

At a high level, the iiONS system can integrate with electronic medical record (EMR) systems to acquire, collect, or otherwise obtain data related to patients and claims. For example, the iiONS system may be able to access, via respective application programming interfaces (APIs), datastores managed by different medical practices to obtain information regarding patients, healthcare professionals, and healthcare facilities. As further discussed below, this information could additionally or alternatively be provided to the iiONS system via, for example, email. This data can be stored in central database tables maintained by the iiONS system. The central database tables may be reflected in user interfaces (or simply “interfaces”) that are generated by the iiONS system. As further discussed below, these interfaces may be representative of a “portal” that can be used by analysts employed by a processing service for internal processing of claims.

Embodiments may be described in the context of computer-executable instructions for the purpose of illustration. However, aspects of the technology can be implemented via hardware, firmware, software, or any combination thereof. As an example, a network-accessible platform that is representative of the iiONS system may be configured to automatically parse the content and attachments of incoming emails to extract, derive, or infer information regarding a claim to be submitted on behalf of a healthcare professional or facility. This information can be “packaged” in a consistent format that is suitable for submittal to an insurer. Then, the network-accessible platform may continually monitor progress of the claim so as to inform stakeholders of its status. For example, the network-accessible platform may notify the healthcare professional or facility when the claim is submitted or approved. As another example, the network-accessible platform may reinitiate communication with the insurer responsive to a determination that no action has occurred within a predetermined interval of time (e.g., 7, 14, or 30 days).

### Terminology

References in this description to “an embodiment,” “one embodiment,” or “some embodiments” mean that the particular feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” or any variant thereof is intended to include any connection or coupling between two or more elements, either direct or indirect. The connection or coupling can be physical, logical, or a combination thereof. For example, devices may be electrically or communicatively coupled to one another despite not sharing a physical connection.

The term “module” may refer broadly to a component implemented via software, firmware, or hardware. Modules are typically functional components that generate data or other output(s) based on specified input(s). A module may be self-contained. A computer program may include one or more modules. Thus, a computer program may include multiple modules responsible for completing different tasks or a single module responsible for completing all tasks.

When used in reference to a list of multiple items, the word “or” is intended to cover all of the following interpretations: any of the items in the list, all of the items in the list, and any combination of items in the list.

The sequences of steps performed in any of the processes described herein are exemplary. However, unless contrary to physical possibility, the steps may be performed in various sequences and combinations. For example, steps could be added to, or removed from, the processes described herein. Similarly, steps could be replaced or reordered. Accordingly, descriptions of any processes are intended to be open-ended.

Overview of iiONS System

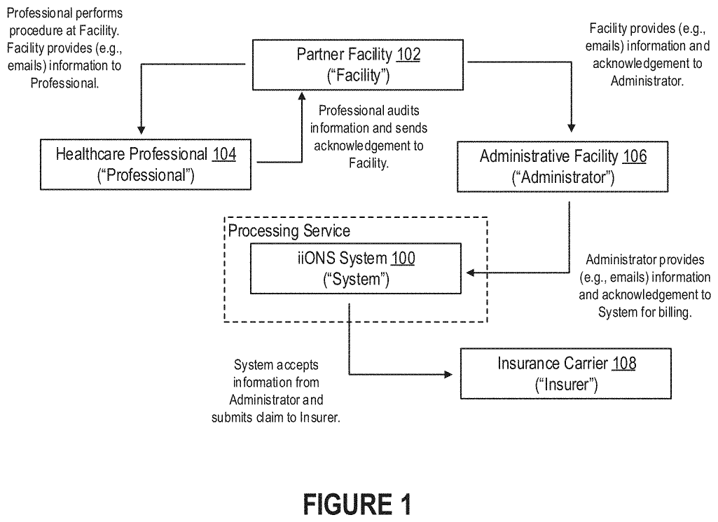

FIG. 1 includes a high-level diagrammatic illustration of a process in which services provided by a healthcare professional 104 at a partner facility 102 are billed to an insurer 108 by the iiONS system 100. At a high level, the iiONS system 100 may be responsible for coordinating billings between a healthcare professional 104, administrative facility 106, and insurer 108 in connection with a service provided at a partner facility 102.

Assume, for example, that the healthcare professional 104 performs a procedure involving a patient at the partner facility 102. Note that while the process shown in FIG. 1 is described in the context of a healthcare professional seeking payment for services rendered, the process is similarly applicable to, for example, seeking payment for equipment that is used by the healthcare professional 104. Examples of healthcare professionals include physicians, specialists, pharmacists, and nurses.

After the procedure is completed, the partner facility 102 can provide information regarding the procedure to the healthcare professional 104. For example, the partner facility 102 may transmit a “facility packet” to the healthcare professional 104 over email. The term “facility packet” may refer to a collection of documents that relate to the procedure performed by the healthcare professional 104 at the partner facility 102. As another example, the partner facility may transmit a communication (e.g., a message or email) to the healthcare professional 104 that includes a link to the facility packet. In such embodiments, the facility packet may be accessible through a network-accessible interface to which the link leads. Through the network-accessible interface, the healthcare professional 104 may be able to download the facility packet. Normally, facility packets are provided to healthcare professionals within 7-10 days after services are provided.

Thereafter, the healthcare professional 104 can audit the information included in the facility packet and then send an acknowledgement to the partner facility 102. As an example, the facility packet may include a form (also referred to as a “checklist”) that the healthcare professional 104 may use to audit the information included in the facility packet. To indicate that she agrees with the information included in the facility packet, the healthcare professional 104 may provide the checklist to the partner facility 102. For example, the healthcare professional 104 could email the checklist to the partner facility 102, or the healthcare professional 104 could upload the checklist to the partner facility 102 through a network-accessible interface. Normally, acknowledgement by the healthcare professional 104 is requested within 21 days after services are provided.

The partner facility 102 can then provide the facility packet and checklist to an administrative facility 106. In some embodiments, the administrative facility 106 is the partner facility 102. In other embodiments, the partner and administrative facilities 102, 106 are different facilities. For example, the administrative facility 106 could be a healthcare center comprised of multiple facilities (e.g., hospitals, clinics, or pharmacies) where services can be provided, while the partner facility 102 may be a single one of those multiple facilities. Generally, the administrative facility 106 performs a cursory review of the facility packet and checklist before forwarding those items onward to the iiONS system 100. For example, the administrative facility 106 may assign a unique identifier to the procedure for tracking purposes and then append the unique identifier to the facility packet and checklist. As another example, the administrative facility 106 may examine the facility packet to ensure that a request for payment has not already been submitted for the procedure. Normally, facility packets and checklists are provided to the iiONS system 100 by administrative facilities within 21 days after services are provided.

After receiving the facility packet and checklist from the administrative facility 106, the iiONS system 100 can extract various information from those items. This information can be stored in a central database table that is maintained by the iiONS system 100. As further discussed below, the central database table may be associated with the procedure, partner facility 102, healthcare professional 104, administrative facility 106, or insurer 108. Thereafter, the iiONS system 100 can submit a claim to the insurer 108 for payment for the procedure performed by the healthcare professional 104 at the partner facility 102. To accomplish this, the iiONS system 100 can provide information that is extracted, derived, inferred, or otherwise obtained from the facility packet and/or checklist to the insurer 108. In effect, the process shown in FIG. 1 is representative of a simplified communication scheme in which information regarding procedures is directed through the iiONS system for processing before requests for payment by insurers. Such an approach not only reduces the likelihood of error (e.g., due to duplicative requests for payment), but also improves the security as fewer requests for payment are submitted to insurers, resulting in fewer opportunities for unauthorized entities (also referred to as “attackers”) to access the sensitive information that is normally included in these requests.

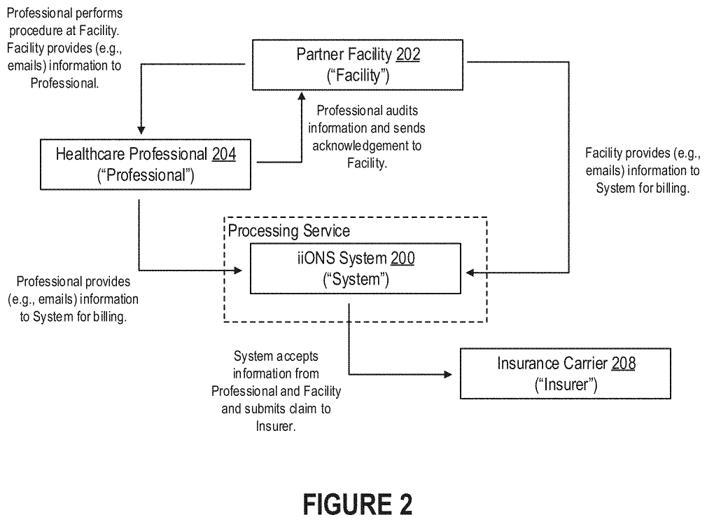

FIG. 2 includes a high-level diagrammatic illustration of another process in which services provided by a healthcare professional 204 at a partner facility 202 are billed to an insurer 208 by the iiONS system 200. In FIG. 1, the healthcare professional 104 provides the checklist to the partner facility 102, which then provides the checklist to the administrative facility 106 for transmission onward to the iiONS System 100. In FIG. 2, however, the healthcare professional 204 provides the checklist directly to the iiONS system 200. This could be done instead of, or in addition to, providing the checklist to the partner facility 202 for acknowledgement purposes. Thus, a healthcare professional could authenticate, verify, or otherwise acknowledge that she provided a service at a partner facility by communicating with the partner facility, iiONS system, or both.

As shown in FIG. 2, the partner facility 202 may provide the facility packet (and checklist, if provided by the healthcare professional 204) to the iiONS system 200 for processing. Because the iiONS system 200 also receives the checklist from the healthcare professional 204, the iiONS system 200 may be able to compare materials received from the healthcare professional 204 against materials received from the partner facility 202. This “dual-track” approach to obtaining information regarding the procedure helps eliminate erroneous and duplicative requests for payment. In a sense, the iiONS system 200 can perform centralized verification by ensuring that the healthcare professional 204 and partner facility 202 have identical accounts of the procedure.

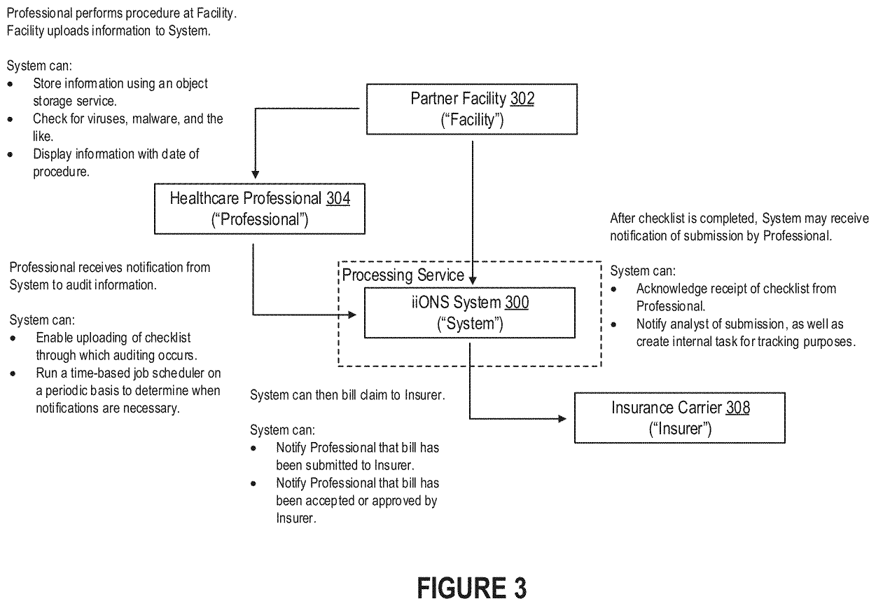

FIG. 3 includes a high-level diagrammatic illustration of an external billing process facilitated by the iiONS system 300. Much like FIG. 2, a healthcare professional 304 may provide a service (e.g., perform a procedure) for a patient at a partner facility 302. Thereafter, the partner facility 302 may provide information regarding the procedure to the healthcare professional 304 and iiONS system 300. For example, the partner facility 302 may provide a facility report to the healthcare professional 304, as discussed above, in order to prompt acknowledgement of the procedure being performed.

Moreover, the partner facility 302 may upload the facility report to the iiONS system 300 via a software extension. A software extension is a type of computer program that is meant to extend the capabilities of another computer program (also referred to as a “base computer program”). The base computer program could be a computer program that already exists on a computing device that is associated with the healthcare professional 304 or partner facility 302. Examples of computing devices include mobile phones, tablet computers, desktop computers, and mobile workstations (also referred to as “computer carts”). Thus, the base computer program may be a mobile application, desktop application, over-the-top (OTT) application, or web browser.

As an example, assume that the healthcare professional 304 indicates, through the base computer program, that the procedure has been completed. In such a scenario, the base computer program or software extension may generate a notification that is intended to prompt the healthcare professional 304 to upload information regarding the procedure. This information could be in the form of a facility packet or checklist. Using the software extension, the healthcare professional 304 may be able to readily transmit this information to the iiONS system 300. From the perspective of the iiONS system 300, this information may appear to come from the healthcare professional 304 or partner facility 302 depending on who is responsible for managing the computing device on which the base computer program (and software extension) executes.

After receiving the facility packet, the iiONS system 300 may perform several different operations as shown in FIG. 3. For example, the iiONS system 300 may store information extracted, derived, or otherwise obtained from the facility packet (or the facility packet itself) using an object storage service. One example of an object storage service is the Simple Storage Service (S3) supported by Amazon Web Services (AWS). The object storage service may create “buckets” into which this information can be stored. The term “bucket” refers to a storage resource that resides on a public cloud infrastructure accessible to multiple users (also referred to as “tenants”). Note, however, that the information stored in a bucket may only be accessible to the corresponding tenant. Accordingly, information stored by the iiONS system 300 using the object storage service may be hashed, hidden, or otherwise obfuscated as a precaution against attackers.

In some embodiments, the iiONS system 300 is programmed to check for viruses, malware, and the like by examining materials uploaded by the healthcare professional 304 and partner facility 302. For example, the iiONS system 300 may utilize a third-party security product through which the materials can be routed for analysis. Then, the iiONS system 300 can determine, based on the outputs produced by the third-party security product, whether any of the materials should be handled in a more sensitive manner (e.g., deleted, quarantined, or scanned). Additionally or alternatively, the iiONS system 300 may employ its own algorithms that have been developed and then trained to identify malicious communications.

As shown in FIG. 3, the iiONS system 300 can submit a request for payment to the insurer 308 based on the information obtained from the healthcare professional 304 or partner facility 302. In embodiments where the iiONS system 300 is accessible through a software extension, additional outreach may be possible to keep the healthcare professional 304 or partner facility 302 apprised of progress. For example, the iiONS system 300 may cause transmission of a notification to the healthcare professional 304 that specifies the request for payment was submitted to the insurer 308, or the iiONS system 300 may cause transmission of a notification to the healthcare professional 304 that specifies the request for payment was accepted or approved by the insurer 308. These notifications may take the form of text messages, email messages, pop-up notifications (e.g., generated by the base computer program), etc. Moreover, the iiONS system 300 may be able to acknowledge receipt of materials from the healthcare professional 304 and partner facility 302.

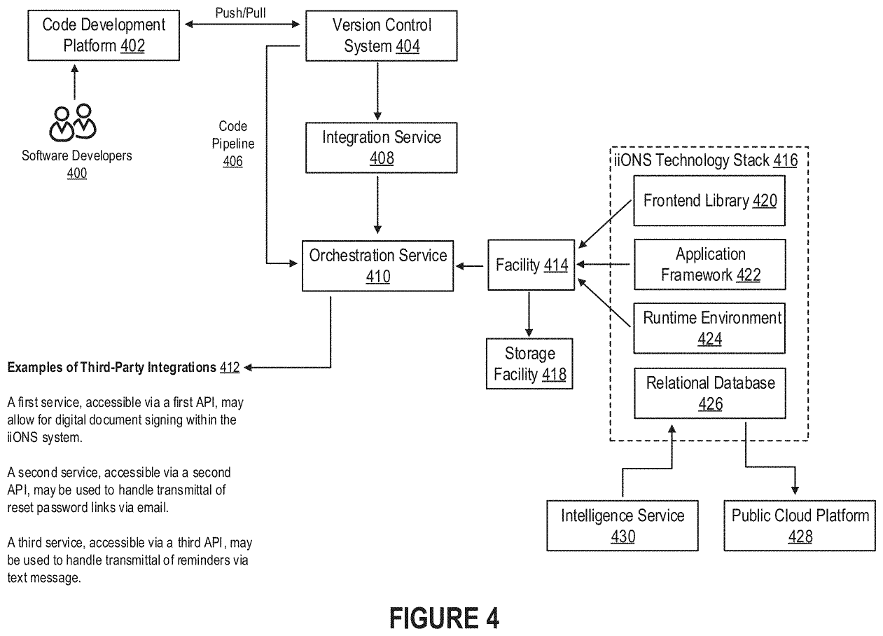

FIG. 4 includes a block diagram illustrating how the iiONS system capable of formulating requests for payment on behalf of healthcare professionals and partner facilities may be implemented. In FIG. 4, software developers 400 interact with a code development platform 402 to push and/or pull software code (or simply “code”) from a version control system 404. An example of a code development platform is GitKraken, and an example of a version control system is Github.

A code pipeline 406 may be used for building, deploying, and then updating the iiONS system. An integration service 408 may be used to compile the source code, run tests, and confirm that the iiONS system is built to specification. Said another way, the software developers 400 may use the integration service 408 to confirm that the iiONS system operates as intended. One example of an integration service is AWS CodeBuild. Meanwhile, the underlying architecture of the iiONS system may be managed by an orchestration service 410. At a high level, the orchestration service 410 may be responsible for deploying and then, when appropriate, scaling the iiONS system. Moreover, the orchestration service 410 may allow the iiONS system to access or utilize various services, including third-party integrations 412 that permit, for example, documents to be digitally signed, passwords to be reset via email, and reminders to be sent via test message. One example of an orchestration service is AWS Elastic Beanstalk.

A facility 414 may be responsible for running the source code (also referred to as the “code base”) that is representative of the technology stack 416 of the iiONS system. The facility 414 could be, for example, the Amazon Elastic Compute Cloud (Amazon EC2). Amazon EC2 is a web service that provides secure, resizable compute capacity in a public cloud infrastructure. As shown in FIG. 4, the technology stack 416 may also utilize a storage facility 418 to store documents received from healthcare professionals and partner facilities.

The iiONS technology stack 416 may include a frontend library 420, application framework 422, runtime environment 424, and relational database 426. The frontend library 420 may allow interfaces to be readily built, rendered, and supported. One example of a frontend library 420 is React, which is an open-source JavaScript library that can be used to create interactive interfaces. The application framework 422 may be operable to connect the interfaces (also referred to as the “frontend” of the iiONS system) to the underlying server-side runtime environment (also referred to as the “backend” of the iiONS system). One example of an application framework is Express.js, which is designed for building web-based applications and APIs. The runtime environment 424 may serve as a processing environment in which the source code of the iiONS system is executed outside of the point of access (e.g., a web browser). One example of a runtime environment is Node.js, which is an open-source, cross-platform JavaScript runtime environment that executes JavaScript code outside of web browsers. The relational database 426 may simply support deployment of the iiONS system. One example of a relational database is MySQL, which is an open-source relational database management system (RDBMS) that can be used to support deployment of cloud-native applications.

As mentioned above, the iiONS technology stack 416 may run on a public cloud architecture 428 (also referred to as a “public cloud platform”). One example of a public cloud platform is AWS. In some embodiments, an intelligence service 430 may be used to gain further insights into the information that is collected, received, or otherwise obtained by the iiONS system. For example, the intelligence service 430 may query data from the relational database 426 in order to produce reports on behalf of a processing service that manages the iiONS system. One example of an intelligence service 430 is Amazon QuickSight.

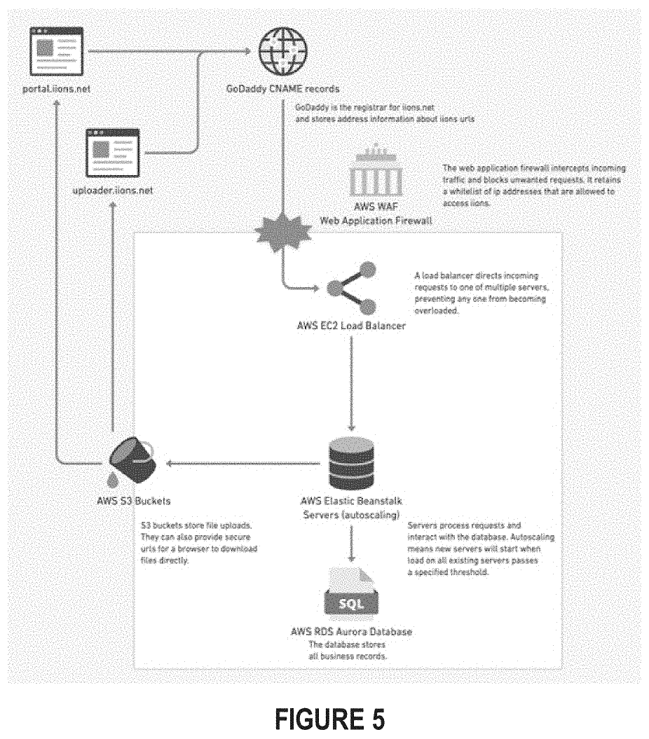

FIG. 5 includes a block diagram illustrating the internal and external dataflows of the iiONS system in one embodiment. In particular, FIG. 5 illustrates the internal and external dataflows of the iiONS system when implemented on AWS using several of the services mentioned above with reference to FIG. 4. Those skilled in the art will recognize that the block diagram shown in FIG. 5 is provided for the purpose of illustration only. The iiONS system could be implemented on another cloud architecture, either public or private, using different services than those shown in FIG. 5.

Advantages of iiONS System

Various benefits of employing the iiONS system to submit requests for payment are provided below. Generally speaking, the iiONS system helps simplify the process of seeking payment from insurers by serving as a central hub through which requests originate. One benefit to this approach is that requests for payment are homogenized in the sense that requests may be generated, formatted, or transmitted in a consistent manner. This helps to ensure that the necessary information is included in each request for payment. Another benefit to this approach is that most, if not all, requests for payment received by a given insurer may come from the iiONS system. Given the prevalence of spoofing—especially in the payment context—this helps the insurer have confidence that requests for payment are actually legitimate. Simply put, the insurer may receive requests for payment from a single domain (e.g., administrator@iiONS.net) rather than various domains associated with different healthcare professionals and facilities.

### A. Avoiding Medical Coding Errors

Based on current American Medical Association (AMA) terminology, the Healthcare Common Procedure Coding (HCPC) system is one example of the type of coding knowledge that's important to have when entering data pertaining to patients. Even if oversights with coding are innocent mistakes, errors that are subsequently discovered can raise red flags and be considered forms of fraud and abuse. The iiONS system can not only identify erroneous coding mistakes made by healthcare professionals and facilities, but also address those mistakes before submitting requests for payment to insurers.

### B. State of the Art Billing

One of the goals of the Health Insurance Portability and Accountability Act (HIPAA) is to establish and manage electronic transactions related to health care. Providers are also required to keep patient records in electronic form. However, it's not easy to stay current with the software and related technology that is needed to accurately and efficiently record and then maintain (e.g., update) billing information for patients.

Having billing information safely and conveniently housed in a secure system allows requests for redeterminations, when appropriate, if the initial decision involving a claim is not satisfactory. Additionally, the iiONS system can be used to spot instances where an account (e.g., associated with a healthcare professional or facility) was overpaid. If the iiONS system determines that overpayment has occurred, the recoupment process can be quickly initiated to secure the appropriate reimbursement. Also, because at least portions of the iiONS system may be implemented on the Internet, the iiONS system may be readily accessible to healthcare professionals and facilities regardless of geographical location. For example, the iiONS system (and, more specifically, its portals) may be accessible to healthcare professionals and facilities throughout the United States.

### C. Tracking Billing Efforts

One of the challenges with seeking payments from insurers is keeping track of the billing process. Because an important goal of healthcare professionals and facilities is to increase profitability, one of the steps that is often taken is to track the response to initial billing efforts. If instances of late, delayed, or incomplete reimbursements are spotted, the iiONS system may automatically take steps to remedy the situation as quickly as possible. As an example, the iiONS system may track the progress of individual requests for payment by logging communication (e.g., from healthcare professionals and facilities, to insurers) and then prompting follow up when necessary. For instance, the iiONS system may generate alerts (e.g., directed to analysts of a processing service that manages the iiONS system) responsive to determining that no progress has been made within a certain interval of time (e.g., 7, 14, or 21 days).

### D. Minimizing Health Insurance Claim Headaches

Many medical practices deal with a combination of reimbursement processes. Some claims may be handled through Medicare or Medicaid, while other claims may be handled through private insurers. Normally, the general process is largely the same, namely, a claim is submitted and then reviewed before a decision regarding reimbursement is made.

While Medicare and Medicaid have very specific and uniform guidelines for seeking payment, this isn't always the case with private insurers. One of the most common and costly headaches for a medical practice is to have a claim unexpectedly denied by a private insurer. To limit denials of this nature, the iiONS system can discover the reason for the denial and then determine if there is a way to autonomously resolve the issue. For example, the iiONS system may attempt to establish whether a change in formatting is necessary, or whether additional information regarding the service, patient, healthcare professional, or partner facility is necessary. If this isn't possible, then the iiONS system can take steps to file an appeal of the denial. In some instances, a denial can be overturned by simply providing more information.

Similar steps may be taken if claims are underpaid or unfairly delayed. In some embodiments, the iiONS system is programmed to spot potential violations of insurance regulations (e.g., local, state, or federal) pertaining to claims due to tardiness.

### E. Avoiding Patient-Provider Conflicts

As mentioned above, the iiONS system may be configurable so that each medical practice can develop its own approach to submitting information regarding claims. By setting up a reimbursement process that works best for each medical practice, the iiONS system can minimize the need to revert to direct patient billing. One way to accomplish this is independently and individually tracking the status of claims. Being diligent about reimbursement can also make things easier for patients as there will be fewer lingering concerns about whether expenses have been sufficiently covered.

### F. Handling Complex Claims

Not all reimbursements are for simple procedures. For example, if a healthcare professional is running a surgical center, she may routinely perform complex procedures that involve many steps (and thus many services). From the initial diagnostic testing to administering anesthetics to performing the actual procedure, every billable step involved in complex procedures must be properly accounted for so that requests for reimbursements can be submitted in accordance with health insurance guidelines. The iiONS system can assist in this respect by maintaining a comprehensive record of actions taken by healthcare professionals and facilities, as well as a reimbursement status for each of those actions.

### G. Improved Efficiency and Consistency in Billing

On average, healthcare facilities lose approximately five percent of their margin due to denied claims, contract negotiations, and underpayments. Inefficient billing practices have a severe impact on providers of services. While every provider attempts to minimize costs and maximize profit, this is difficult to accomplish. By taking advantage of advancements in technologies, the iiONS system can approach coding and billing in a radically different manner. As an example, advancements in software-implemented automation (e.g., in processing, filtering, and formatting data) may help in reducing the effort needed to seek reimbursement.

### H. Eligibility Verification

On average, medical practices submit approximately 83 claims per day. Combined with the fact that only half of outstanding bills are collected within 30 days, and it becomes clear that a slow process for verifying eligibility can complicate billing procedures. Without an efficient verification process, a medical practice will be at risk of losing revenue through denied claims, billing leakages, and the like. In some embodiments, the iiONS system is able to execute a computer program designed to assist in eligibility verification. As such, the iiONS system may not need to constantly verify each patient. Moreover, the iiONS system may execute another computer program that is designed to check copayment, eligibility, and coinsurance rules in order to easily determine the portion of reimbursements for which patients are responsible.

### I. Artificial Intelligence

Historically, medical coders (or simply “coders”) manually transcribed all of the information regarding the services provided to a patient during a visit. These coders then transcribed this information into an electronic form that was stored in a database managed by a medical practice. Today, healthcare professionals normally use encoders to assist in transcription of this information. However, coders still play an important role due to the likelihood and impact of errors in converting this information into the codes used by health insurance plans.

In an effort to eliminate these errors, the iiONS system may employ artificial intelligence to proficiently identify mistakes and then fix those mistakes. For example, artificial intelligence may be employed as an “oversight mechanism” that reviews the actions taken in accordance with the heuristics and rules programmed in the underlying code. Moreover, artificial intelligence may provide real-time analysis to coders to help improve efficiency. As a result, the rejection rate of bills submitted by the iiONS system are lowered due to fewer errors.

Embodiments of the iiONS system may also use artificial intelligence to:
Convey information regarding claims to, for example, a single color (e.g., green, yellow, red, or black) that is representative of the workflow that each claim can take;
Determine which documents are required to process a claim and then obtain or request those documents; and
Populate fields in forms (e.g., the HCFA and UB-04 claim forms) in an automated manner so as to reduce the likelihood of error.
   Operating Procedures for iiONS System

As mentioned above, the iiONS system may be managed by a processing service that can assist healthcare professionals and facilities in seeking reimbursement from insurers. At a high level, the processing service can review patients' contracts, health codes, billing rates, and equipment in order to help medical practices develop a personalized plan for seeking reimbursement. Below, examples of process for billing, tasking, appealing, and suspending claims are described. These examples have been provided solely for the purpose of illustration.

### A. Billing

Assume, for example, that an analyst employed by a processing service receives an email that includes one or more documents pertaining to a procedure involving a patient. That email may be addressed to an email address that is associated with the processing service (and thus the iiONS system). The analyst can then open the email and begin reviewing the documents to initiate the process for accepting a new patient file. Moreover, the analyst may create a patient folder through the iiONS system in which template folders for patient information, claims, correspondence, or appeals can be stored. Each document may be uploaded to the appropriate template folder within the patient folder by the analyst.

Thereafter, the analyst may open an audit checklist (or simply “checklist”) that can be used to facilitate review for accuracy and completeness of the documents. For example, the checklist may prompt the analyst to confirm that one or more of the following are present: copy of insurance card, copy of driver license, patient information (e.g., demographic information), copy of invoice, procedure summary (e.g., medical records such as surgery report, post-operation report, etc.), and the like. These documents can be kept in a readily available place to prepare for entering the patient into the iiONS system.

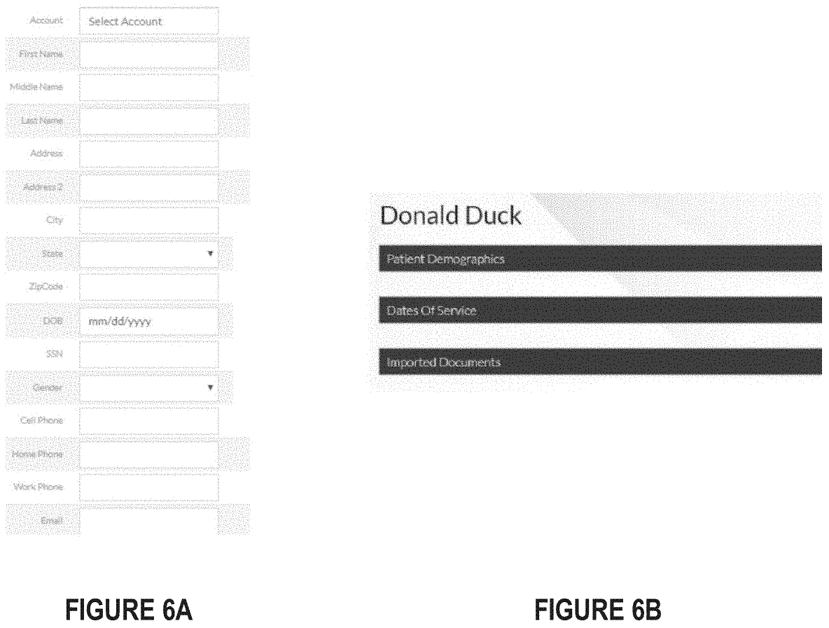

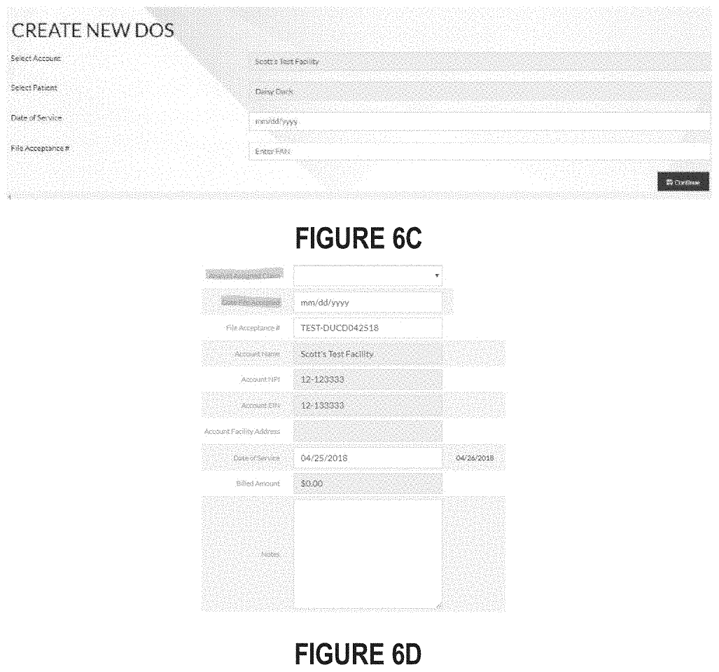

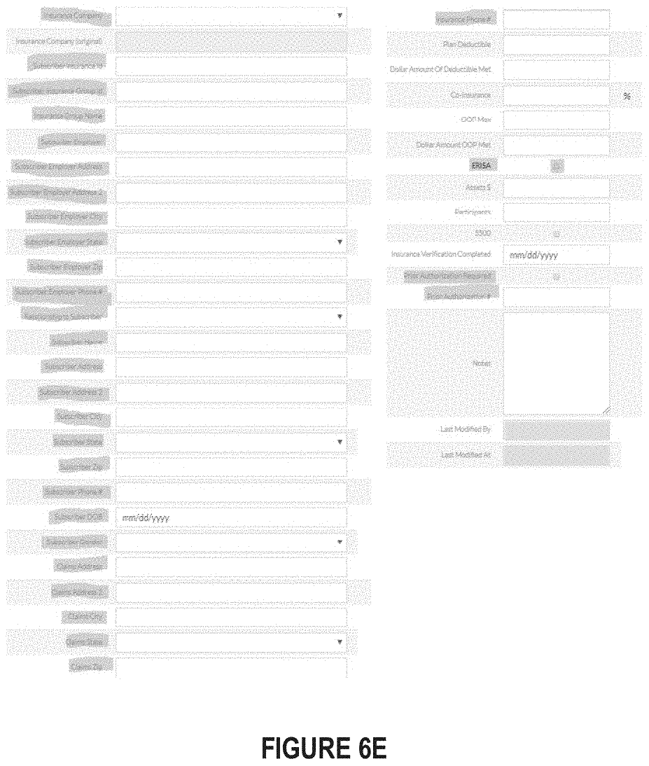

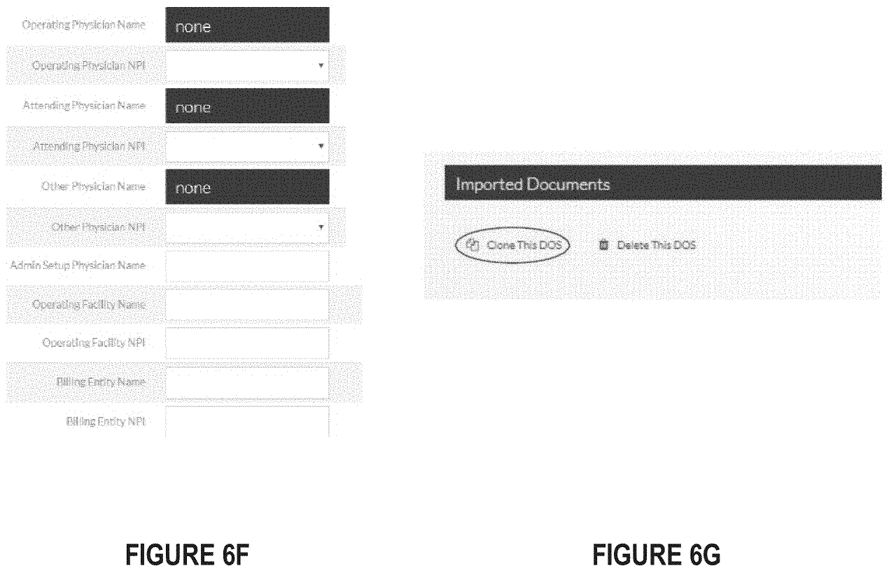

Before entering the patient into the iiONS system, the analyst may check to see whether the patient already exists in the system, for example, by typing the name in a search bar located along the top of a landing page generated by the iiONS system. If the search returns a contact with the same name, then the analyst may select the record and then specify another piece of information (e.g., the date of birth) to verify that the contact corresponds to the patient. However, if no records are returned in response to the search, then the analyst may select a digital element labelled “Add New Patient” that is located along the top of the landing page. Selecting this digital element may cause display of the interface shown in FIG. 6A. As shown in FIG. 6A, the analyst may use information that is extractable, derivable, or inferable from the documents to populate fields such as account, first name, middle name, last name, address, city, state, zip code, birthdate, social security number (SSN), gender, cell phone number, home phone number, work phone number, email and address. After saving this information, a patent contact will be created as shown in FIG. 6B.

After the patient contact is created, the analyst may be able to create a date of service (DOS) by selecting a digital element labelled “Add New DOS”). Such action may result in the display of the interface shown in FIG. 6C. The analyst can use information included in the documents, as well as the patient contact, to populate fields that specify the account, patient, date of service, and file number. The file number may indicate the type of service that was received. For example, the file number may specify whether the service pertained to anesthesia, surgery, radiology, etc. In some embodiments, further information may be requested as shown in FIG. 6D. Such information may include the assigned analyst (i.e., the analyst's name), billed amount, and date of file acceptance from provider. Moreover, the analyst could be prompted to upload the documents directly. Thus, the analyst may attach documents received from the provider to the record of the service that was provided.

Then, the analyst can continue to the interface shown in FIG. 6E. Through this interface, the analyst can enter information related to the insurer to whom the claim will be submitted. This information can include the name of the insurer, subscriber identifier, subscriber group identifier, subscriber employer, subscriber employer address, subscriber employer city, subscriber employer state, subscriber employer zip, subscriber employer phone number, relationship to subscriber, subscriber name, subscriber address, subscriber phone number, subscriber date of birth, and subscriber gender.

Moreover, the analyst may populate the interface shown in FIG. 6F with information regarding the healthcare professional responsible for providing the service. As an example, if the patient underwent a surgical procedure, then the analyst may enter the operating physician name, attending physician name, other physician name(s), operating facility name, and billing entity name.

Note that, in some embodiments, the analyst may simply be able to “clone” existing DOSs. Assume, for example, that the analyst receives an email with documents from which she can infer that the corresponding patient has completed an annual physical examination. Rather than complete a new DOS from scratch, the analyst may “clone” an existing DOS as shown in FIG. 6G. For instance, the analyst may create an identical copy of the existing DOS for the most recent physical examination and then make changes as necessary. Since the existing DOS may have already been approved by the insurer for reimbursement, the analyst can use the existing DOS as a template to limit the likelihood of rejection.

Comparable interfaces may be used to generate a claim on behalf of a healthcare facility rather than a healthcare professional.

### B. Tasking

In some scenarios, analysts may want to perform additional actions before or after claims have been submitted using the iiONS system. Normally, these actions are performed upon receiving documents from the patient, healthcare professional, healthcare facility, or insurer related to the claim.

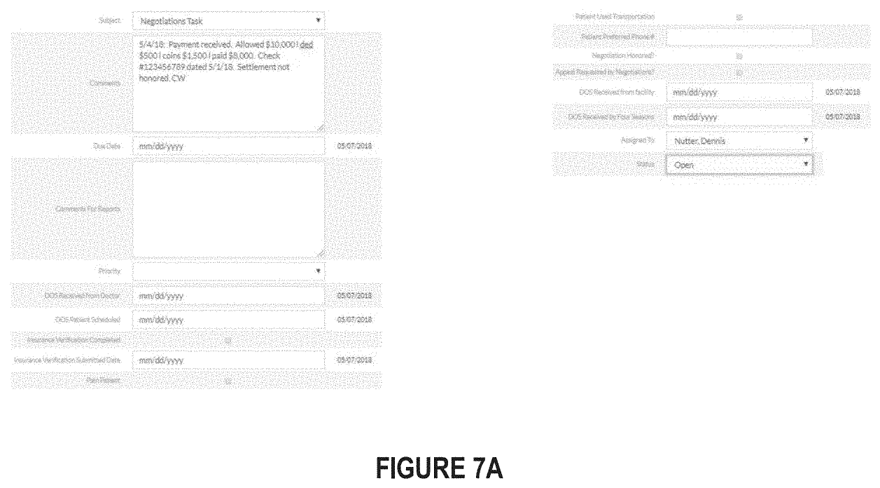

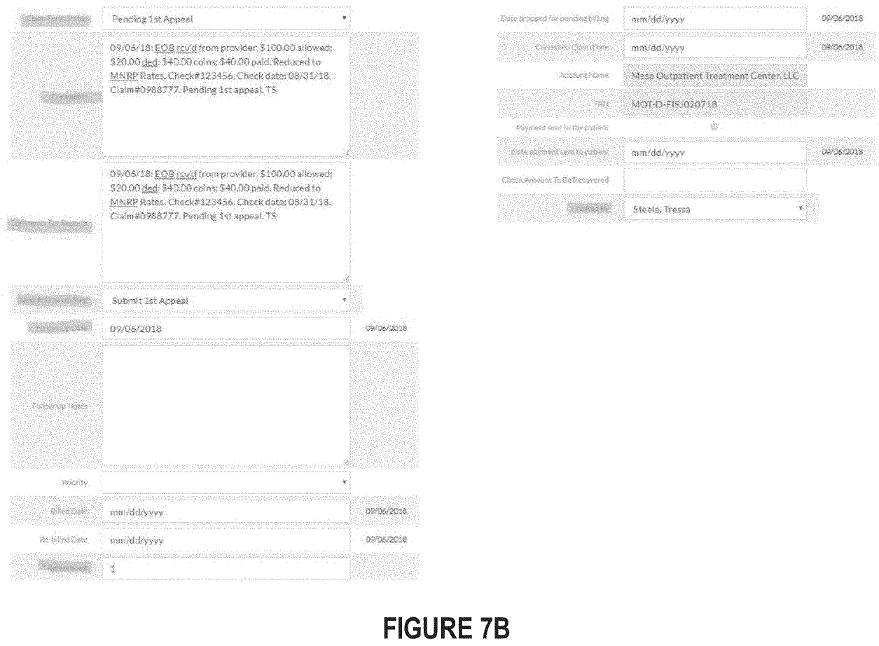

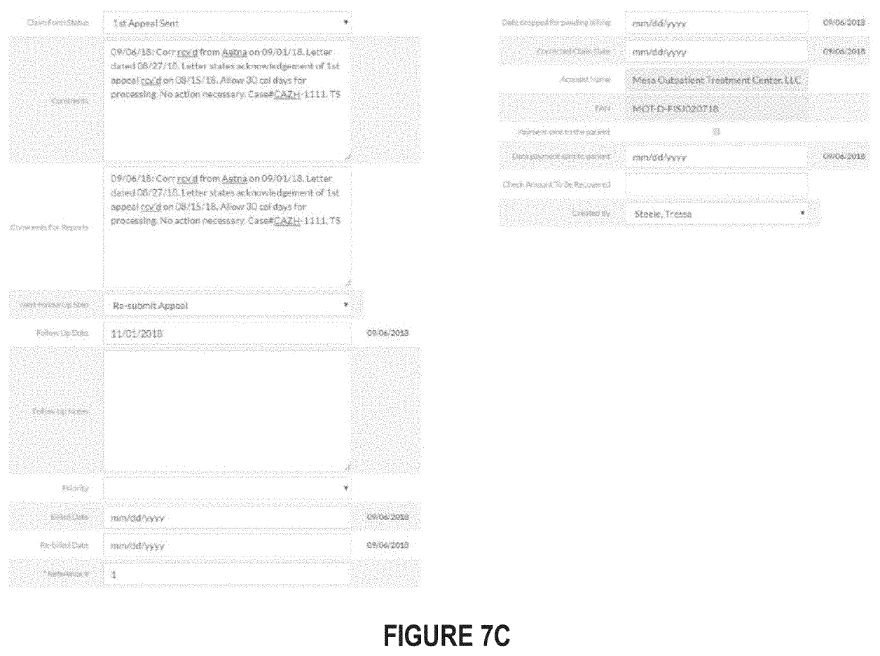

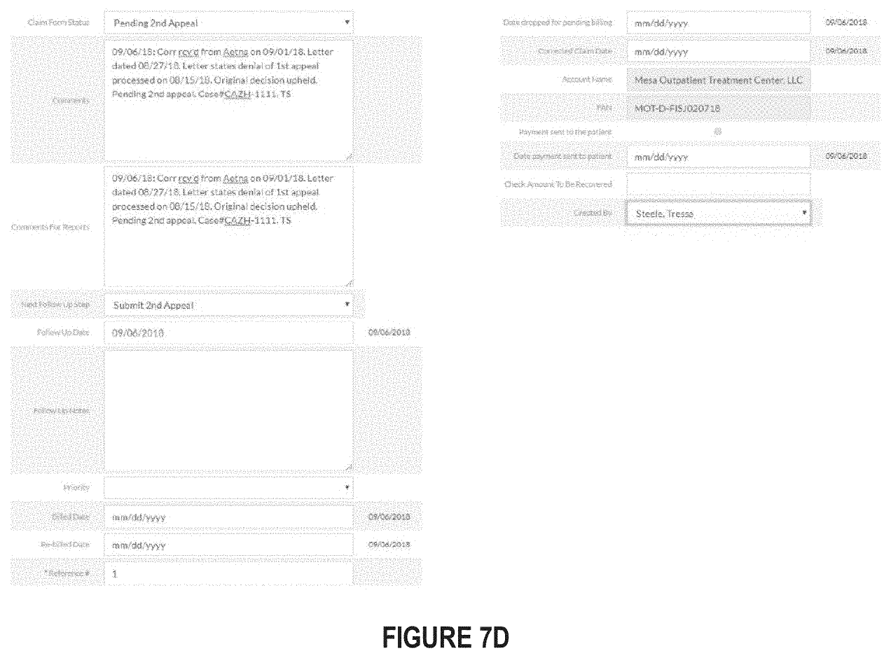

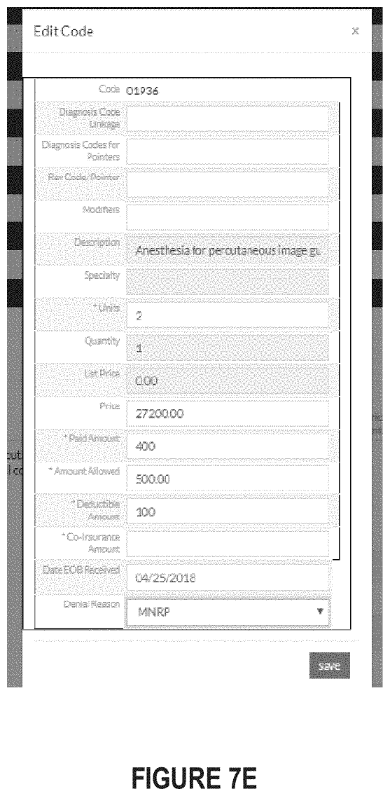

As an example, an analyst may be responsible for asking an insurer to initiate negotiations over a claim. To do this, the analyst may initiate the iiONS system and then select the appropriate DOS. After selecting the DOS, the analyst may open a new task to access the interface shown in FIG. 7A. As shown in FIG. 7A, the analyst may select “Negotiation Task” as the subject and then complete the remaining fields before assigning the negotiation task. Here, the negotiation task is assigned to Dennis Nutter.

As another example, an analyst may wish to update the status of a claim based on, for example, incoming correspondence from the healthcare professional, healthcare facility, or insurer. To do this, the analyst may initiate the iiONS system and then select the appropriate DOS. After selecting the DOS, the analyst may use the interface shown in FIG. 7B to specify updates. Here, the analyst has provided additional comments regarding the state of the claim pending a first appeal. FIG. 7C includes an interface that illustrates how the analyst may update a DOS after receiving an acknowledgement letter from an insurer, while FIG. 7D includes an interface that illustrates how the analyst may update a DOS after receiving a denial letter from an insurer.

As another example, an analyst may wish to record that a payment has been made so as to update a stakeholder interested in the outcome of the claim. The stakeholder could be the patient, healthcare professional, or healthcare facility. To do this, the analyst may initiate the iiONS system and then select the appropriate DOS. After selecting the DOS, the analyst may use the interface shown in FIG. 7E to record that a payment has been made.

### C. Appealing

Appeals may be initiated by the iiONS system if a claim has not been sufficiently paid. Said another way, the iiONS system may initiate an appeal if the insurer reimburses less than a predetermined portion (e.g., 75, 85, or 90 percent) of the total amount sought. Appeals may be either automatically sent by the iiONS system or manually sent by an analyst using the iiONS system on behalf of the healthcare professional or facility seeking reimbursement.

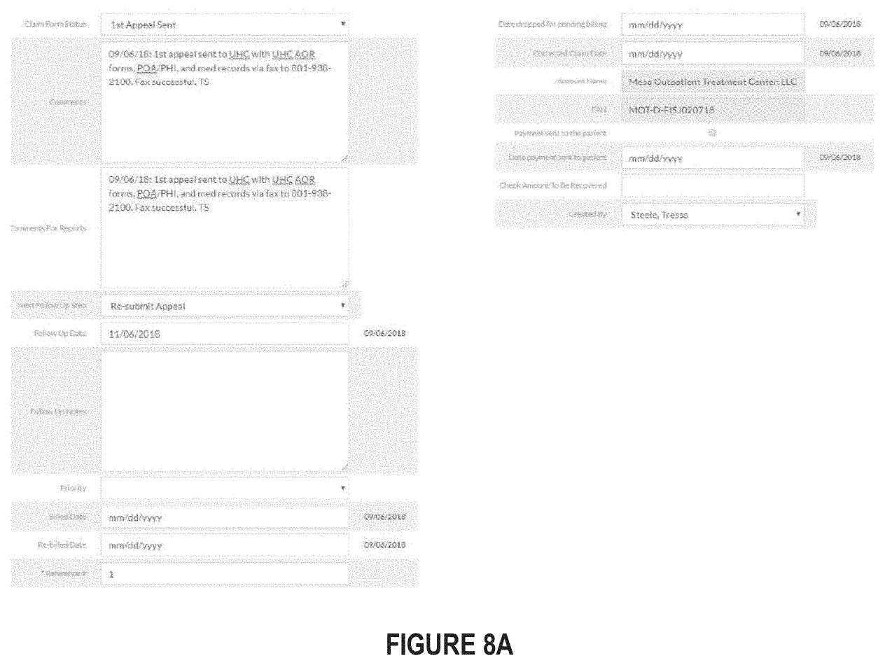

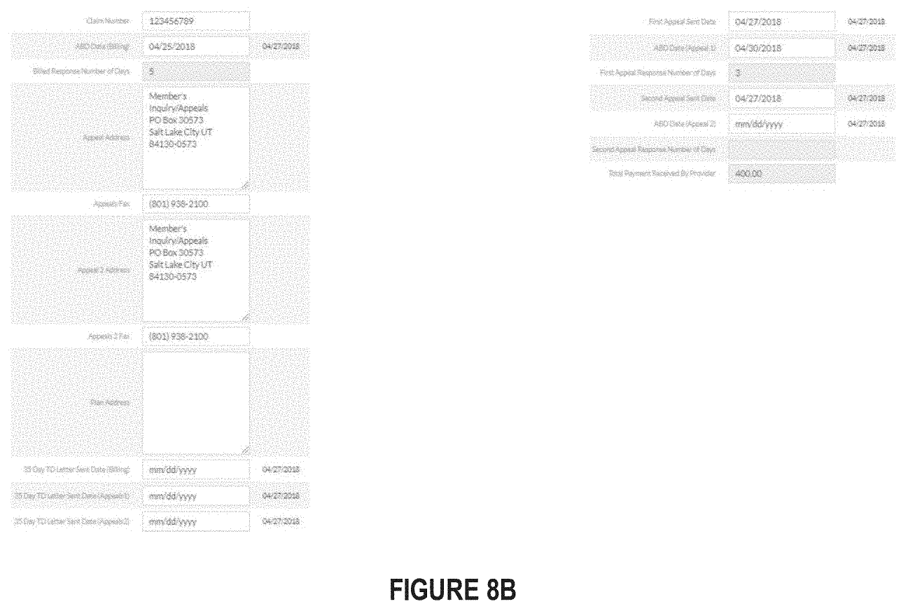

As appeals are initiated, analysts may wish to provide comments as shown in FIG. 8A. These comments may be helpful in providing context regarding the appeal. To initiate an appeal, analysts may use the interface shown in FIG. 8B. Note that multiple appeals could be initiated for a single DOS. In FIG. 8B, for example, a second appeal is being initiated due to denial of a first appeal with insufficient payment. Normally, appeal resubmissions will be an exact copy of the original appeal. As such, the iiONS system may automatically populate portions of the interface shown in FIG. 8B responsive to receiving input indicative of a request to initiate a second appeal. However, the analyst may be permitted to add, remove, or adjust information as necessary. Thus, the analyst may be able to easily add more information if the original appeal is rejected, for example, due to inadequate information.

### D. Suspending

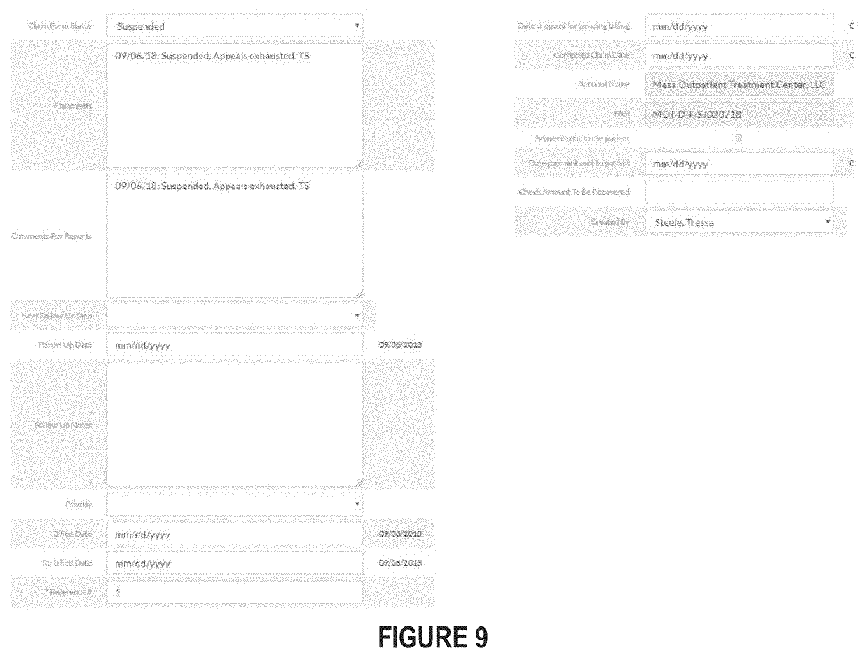

Claims may be suspended if the iiONS system determines that (i) a predetermined number (e.g., 1, 2, or 3) of appeals have been exhausted or (ii) more than a predetermined portion (e.g., 75, 85, or 90 percent) of the total amount sought has been reimbursed. This is normally done automatically by the iiONS system. However, analysts may also be permitted to suspend claims using the iiONS system. FIG. 9 includes an example of an interface through which notes regarding suspension can be added.

### Methodologies for Adjudicating Claims

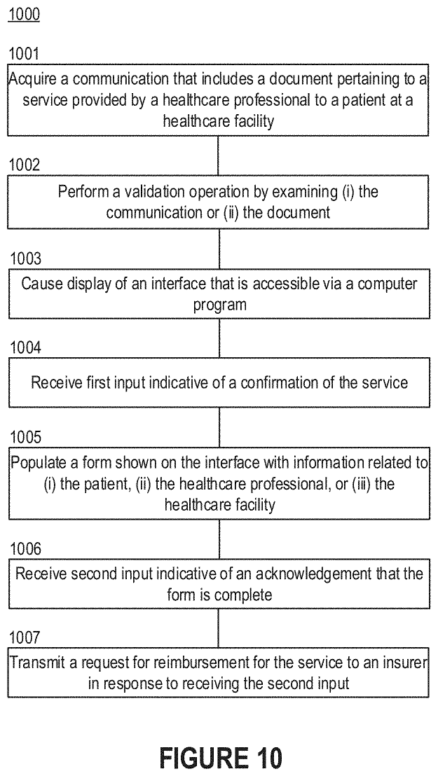

FIG. 10 includes a flow diagram of a process 1000 for submitting a request for reimbursement to an insurer for a service provided to a patient. Note that the term “request for reimbursement” may be interchangeable with the terms “claim,” “bill,” and “invoice.” Initially, an iiONS system can acquire a communication that includes a document pertaining to a service provided by a healthcare professional to a patient at a healthcare facility (step 1001). Normally, this communication is in the form of an email message to which the document is secured as an attachment. However, the document could alternatively be uploaded to the iiONS system through a network-accessible interface as discussed above.

Thereafter, the iiONS system may perform a validation operation by examining (i) the communication or (ii) the document (step 1002). At a high level, the validation operation may be performed to ensure that the communication is legitimate. As an example, the iiONS system may scan the communication and document to check for viruses, malware, and the like. As another example, the iiONS system may compare the source against a list of known “good” contacts from which the iiONS system is permitted to receive communications. This list may be referred to as a “whitelist.” Thus, the communication may be validated as authentic responsive to a determination that the communication originated from an email address known to be associated with a healthcare professional with whom the iiONS system has a preexisting relationship. Similarly, the communication may be validated as authentic responsive to a determination that the communication originated from an email address known to be associated with a healthcare facility with which the iiONS system has a preexisting relationship.

Then, the iiONS system can cause display of an interface that is accessible via a computer program (step 1003). In some embodiments the interface is accessible via a web browser, while in other embodiments the interface is accessible via a mobile application, desktop application, or OTT application. Normally, the computer program is executed by a computing device that is associated with an analyst employed by a processing service. As mentioned above, the processing service may be responsible for processing claims on behalf of providers (e.g., healthcare professionals and facilities).

The iiONS system can then receive first input indicative of a confirmation of the service (step 1004). This first input may be provided through the interface. For example, the analyst may select the service from a drop-down menu of available services for which reimbursement can be sought. As another example, the analyst may type a description of the service as determined based on the communication or document. Alternatively, the iiONS system may automatically determine the service that was provided based on an analysis of the communication or document. In such embodiments, the analyst may simply be responsible for confirming or correcting inferences (also referred to as “predictions”) made by the iiONS system. Moreover, in some embodiments, the iiONS system is configured to populate a form shown on the interface with information related to (i) the patient, (ii) the healthcare professional, or (iii) the healthcare facility (step 1005). The iiONS system may extract or derive this information from the communication or document, or the iiONS system may extract or derive this information from records (also referred to as “profiles”) associated with the patient, healthcare professional, and healthcare facility. Thus, the iiONS system may automatically populate at least some fields included in the form so as to lessen the likelihood that the claim will be rejected due to erroneous information, improper format, etc.

Note that the iiONS system may enable the analyst to readily adjust the content of the fields included in the form, regardless of whether those fields are automatically populated by the iiONS system. Thus, the iiONS system may enable the analyst to alter the information populated into the form by the iiONS system. Moreover, the iiONS system may enable the analyst to add new information to the form and remove existing information to the form. These changes may be tracked by a machine learning algorithm executed by the iiONS system in order to better understand the types of changes made by the analyst. Said another way, the iiONS system may attempt to learn from the changes that are made by the analyst to improve its ability to populate fields.

Thereafter, the iiONS system may receive second input indicative of an acknowledgement from the analyst that the form is complete (step 1006). Like the first input, the second input may be provided through the computer program. For example, the analyst may indicate that the form is complete by selecting a digital element labelled “Save” or “Submit.” The iiONS system can then transmit a request for reimbursement for the service to an insurer in response to receiving the second input (step 1007). This request may include at least some of the information included in the form that is visible on the interface. Accordingly, the iiONS system may use the information included in the form—both input by the analyst and populated by the iiONS system—to generate a claim for the service provided to the patient. Not all of the information included in the form is necessarily included in the request for reimbursement, however. Some of this information may by used by the iiONS system, for example, for tracking or allocating purposes. As an example, the form may include a unique identifier that is associated with the patient, healthcare professional, or healthcare facility and is used by the iiONS system to track claims having the same identifier.

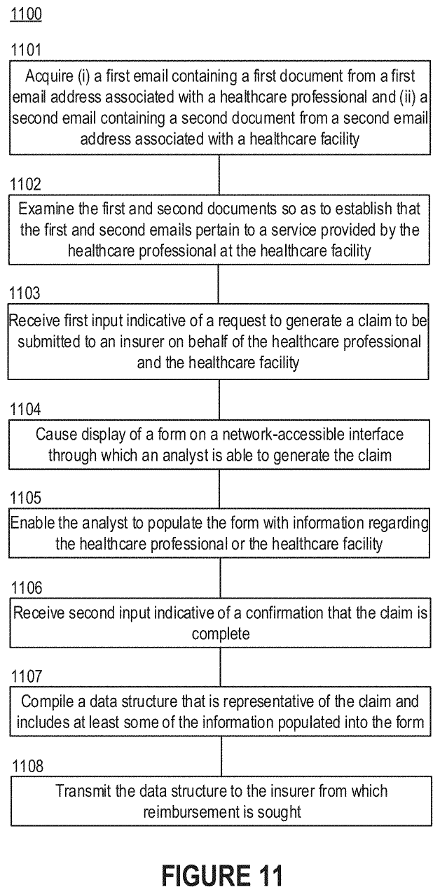

FIG. 11 includes a flow diagram of a process 1100 for generating a claim on behalf of a healthcare professional and facility. Initially, an iiONS system can acquire (i) a first email containing a first document from a first email address associated with a healthcare professional and (ii) a second email containing a second document from a second email address associated with a healthcare facility (step 1101). The first and second emails may be directed to a third email address that is associated with the iiONS system. As an example, the third email address may have a domain (e.g., invoicing@ProcessingService.com) that is representative of the processing service responsible for managing the iiONS system.

The iiONS system can then examine the first and second documents so as to establish that the first and second emails pertain to a service provided by the healthcare professional at the healthcare facility (step 1102). This may involve automated analysis of the content of the first and second documents by the iiONS system. For example, the iiONS system may determine whether the first and second documents are representative of identical copies of a single document. As another example, the iiONS system may compare information extracted from the first document to information extracted from the second document to establish that both documents relate to the same service. Such information could include patient name, date of service, name of healthcare professional, name of healthcare facility, description of service, code for service, and the like.

Then, the iiONS system may receive first input indicative of a request to generate a claim to be submitted to an insurer on behalf of the healthcare professional and the healthcare facility (step 1103). For example, in embodiments where an analyst is able to interact with the iiONS system via an interface, the first input may be representative of a selection, by the analyst, of a digital element labelled “New DOS” or “Generate Claim.” Normally, claims are not generated until some indication is received by the iiONS system that an analyst is ready to proceed. However, in some embodiments, the iiONS system may automatically initiate claim generation responsive to determining that all of the necessary information and documentation is available. The iiONS system may reach this determination by comparing the documents to a programmed data structure that indicates, for various services, what documents are needed to complete a valid claim.

The iiONS system can then cause display of a form on a network-accessible interface through which an analyst is able to generate the claim (step 1104). Step 1104 of FIG. 11 may be similar to step 1003 of FIG. 10. Moreover, the iiONS system may enable the analyst to populate the form with information regarding the healthcare professional or the healthcare facility (step 1105). This information may be extractable, derivable, or inferable from the first and second documents, or this information may be obtainable from profiles maintained by the iiONS system for the healthcare professional and healthcare facility.

Thereafter, the iiONS system may receive second input indicative of a conformation that the claim is complete (step 1106). In some embodiments, the iiONS system simply waits until the analyst indicates that the claim is complete. In other embodiments, the iiONS system may monitor the activities of the analyst to infer whether the claim is complete. For example, upon determining that all of the fields in the form have been filled out, the iiONS system may generate a prompt that asks the analyst to indicate whether the claim is complete. After receiving the second input, the iiONS system can compile a data structure that is representative of the claim and includes at least some of the information populated into the form (step 1107) and then transmit the data structure to the insurer from which reimbursement is sought (step 1108).

Other steps could also be performed.

For example, the first and second documents (or information extracted or derived from those documents) may be stored in a profile that is associated with the patient, healthcare professional, healthcare facility, or service itself. Thus, the iiONS system may generate a coherent record of the service even though documents are received from different sources (e.g., the healthcare professional and facility). As discussed above, these documents can also be stored in a secure manner, thereby improving the security of a transaction that has historically been a target for fraud.

As another example, the iiONS system may be programmed to notify stakeholders of the progress of the claim. This can be done in a periodic or ad hoc manner. For example, the iiONS system may cause transmission of a notification to the healthcare professional or healthcare facility so as to indicate that the claim has been submitted to the insurer. Similarly, notifications may be transmitted to notify the healthcare professional or healthcare facility of appeals, underpayments, delays, and the like. As mentioned above, these notifications may be in the form of emails. Thus, the iiONS system may transmit notifications to the email addresses from which documents were originally received from the healthcare professional and healthcare facility.

As another example, the iiONS system may employ artificial intelligence to autonomously review the information included in the data structure (and thus the conduct of the analyst). More specifically, the iiONS system may execute an artificial intelligence algorithm that is designed to ensure that the data structure to be provided to the insurer complies with guidelines of an insurance plan through which the reimbursement is sought. This “forward looking” analysis can be helpful in proactively identifying and then addressing minor issues that might halt or slow reimbursement.

As another example, the iiONS system may assist the analyst in completing the form by automatically populating some fields. For example, after acquiring the first and second emails from the healthcare professional and healthcare facility, the iiONS system may extract the first and second documents and then perform a text recognition operation on those document so as to convert the content into machine-encoded text. Then, the iiONS system can create, for the healthcare professional, a first data structure that includes information derived from the machine-encoded text corresponding to the first document. Similarly, the iiONS system can create, for the healthcare facility, a second data structure that includes information derived from the machine-encoded text corresponding to the second document. The first and second data structures may be used to facilitate population of the information into various fields of the form.

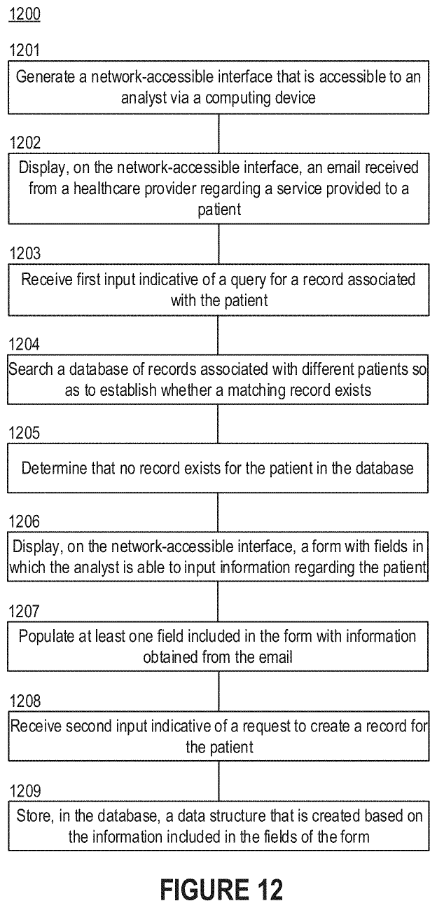

FIG. 12 includes a flow diagram of a process 1200 for creating a record for a patient to whom a service has been provided for which reimbursement will be sought. Initially, an iiONS system can generate a network-accessible interface that is accessible to an analyst via a computing device (step 1201). Then, the iiONS system can display, on the network-accessible interface, an email received from a healthcare provider regarding a service provided to a patient (step 1202). The healthcare provider could be a healthcare professional or a healthcare facility. In some embodiments, the iiONS system displays a visual representation of the email on the network-accessible interface rather than the email itself. For example, the iiONS system may present information related to the email, such as sender name, sender email address, time of transmission, and subject, as well as any documents that were secured as attachments.

Thereafter, the iiONS system may receive first input indicative of a query for a record (also referred to as a “contact”) associated with the patient (step 1203). For example, the analyst may enter the name of the patient into a search field included on the network-accessible interface. The name of the patient could be determined based on the contents of the email or its attachments.

The iiONS system can then search a database of records associated with different patients so as to establish whether a matching record exists (step 1204). As discussed above, if a matching record exists, then the iiONS system may prompt the analyst to enter additional information (e.g., a date of birth) for verification purposes before permitting the analyst to access the matching record. However, the iiONS system may determine that no record exists for the patient in the database (step 1205). This may be the case if this is the first time that the patient has sought treatment under a new health insurance plan. In such a scenario, the iiONS system can display, on the network-accessible interface, a form with fields in which the analyst is able to input information regarding the patient (step 1206). In some embodiments, the iiONS system is able to populate at least one field included in the form with information obtained from the email or its attachments (step 1207).

Thereafter, the iiONS system may receive second input indicative of a request to create a record for the patient (step 1208). For example, after filling out the fields in the form, the analyst may select a digital element labelled “Save Record” or “Store Record.” The iiONS system can then store, in the database, a data structure that is created based on the information included in the fields of the form (step 1209). While this approach to creating patient records appears methodical, it lessens the likelihood of erroneous data being entered and increases the speed with which claims can subsequently be generated for patients.

### Processing System

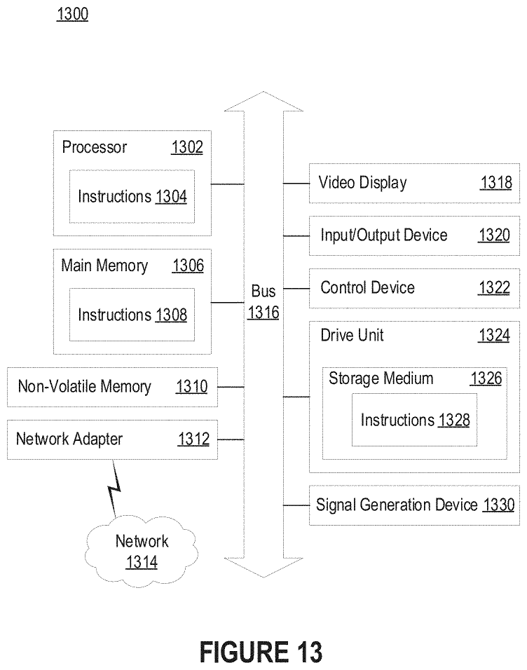

FIG. 13 is a block diagram illustrating an example of a processing system 1300 in which at least some operations described herein can be implemented. For example, components of the processing system 1300 may be hosted on a computing device that executes the iiONS system. As another example, components of the processing system 1300 may be hosted on a computing device through which an individual (e.g., an analyst) may be able to interact with the iiONS system.

The processing system 1300 may include a central processing unit (“processor”) 1302, main memory 1306, non-volatile memory 1310, network adapter 1312 (e.g., a network interface), video display 1318, input/output devices 1320, control device 1322 (e.g., a keyboard or pointing device), drive unit 1324 including a storage medium 1326, and signal generation device 1330 that are communicatively connected to a bus 1316. The bus 1316 is illustrated as an abstraction that represents one or more physical buses or point-to-point connections that are connected by appropriate bridges, adapters, or controllers. The bus 1316, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I²C) bus, or an Institute of Electrical and Electronics Engineers (IEEE) standard 1394 bus (also referred to as “Firewire”).

The processing system 1300 may share a similar processor architecture as that of a desktop computer, tablet computer, mobile phone, game console, music player, wearable electronic device (e.g., a watch or fitness tracker), network-connected (“smart”) device (e.g., a television or home assistant device), virtual/augmented reality systems (e.g., a head-mounted display), or another electronic device capable of executing a set of instructions (sequential or otherwise) that specify action(s) to be taken by the processing system 1300.

While the main memory 1306, non-volatile memory 1310, and storage medium 1326 are shown to be a single medium, the terms “machine-readable medium” and “storage medium” should be taken to include a single medium or multiple media (e.g., a centralized/distributed database and/or associated caches and servers) that store one or more sets of instructions 1328. The terms “machine-readable medium” and “storage medium” shall also be taken to include any medium that is capable of storing, encoding, or carrying a set of instructions for execution by the processing system 1300.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 1304, 1308, 1328) set at various times in various memory and storage devices in an electronic device. When read and executed by the processors 1302, the instruction(s) cause the processing system 1300 to perform operations to execute elements involving the various aspects of the present disclosure.

Moreover, while embodiments have been described in the context of fully functioning electronic devices, those skilled in the art will appreciate that some aspects of the technology are capable of being distributed as a program product in a variety of forms. The present disclosure applies regardless of the particular type of machine- or computer-readable media used to effect distribution.

Further examples of machine- and computer-readable media include recordable-type media, such as volatile and non-volatile memory devices 1310, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1312 enables the processing system 1300 to mediate data in a network 1314 with an entity that is external to the processing system 1300 through any communication protocol supported by the processing system 1300 and the external entity. The network adapter 1312 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

The network adapter 1312 may include a firewall that governs and/or manages permission to access/proxy data in a network. The firewall may also track varying levels of trust between different machines and/or applications. The firewall can be any number of modules having any combination of hardware, firmware, or software components able to enforce a predetermined set of access rights between a set of machines and applications, machines and machines, or applications and applications (e.g., to regulate the flow of traffic and resource sharing between these entities). The firewall may additionally manage and/or have access to an access control list that details permissions including the access and operation rights of an object by an individual, a machine, or an application, and the circumstances under which the permission rights stand.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method comprising:
acquiring, by a computing device, a communication that includes a document pertaining to a service provided by a healthcare professional to a patient at a healthcare facility;
performing, by the computing device, a validation operation by examining (i) the communication or (ii) the document;
causing, by the computing device, display of an interface that is accessible via a computer program;
receiving, by the computing device, first input indicative of a confirmation of the service, the first input being provided through the computer program;
populating, by the computing device, a form shown on the interface with information related to (i) the patient, (ii) the healthcare professional, or (iii) the healthcare facility;
receiving, by the computing device, second input indicative of an acknowledgement that the form is complete, the second input being provided through the computer program; and
transmitting, by the computing device, a request for reimbursement for the service to an insurer in response to receiving the second input, wherein the request includes at least some of the information included in the form shown on the interface.

2. The method of claim 1, wherein the communication is an email message to which the document is secured as an attachment.

3. The method of claim 1, wherein the computing device is a computer server that is part of a public cloud infrastructure.

4. The method of claim 1, wherein the validation operation is performed so as to ensure that the communication is a legitimate request to submit the claim.

5. The method of claim 1, wherein the validation operation is configured to validate the communication as authentic responsive to a determination that the communication originated from an email address known to be associated with a healthcare professional.

6. The method of claim 1, wherein the validation operation is configured to validate the communication as authentic responsive to a determination that the communication originated from an email address known to be associated with a healthcare facility.

7. The method of claim 1, further comprising:
enabling, by the computing device, an analyst to
alter the information populated into the form through the computer program, and
add new information to the form through the computer program.

8. A non-transitory medium with instructions stored thereon that, when executed by a processor of a computing device, cause the computing device to perform operations comprising:
acquiring
(i) a first email containing a first document from a first email address associated with a healthcare professional, and
(ii) a second email containing a second document from a second email address associated with a healthcare facility;
examining the first and second documents so as to establish that the first and second emails pertain to a service provided by the healthcare professional at the healthcare facility;
receiving first input indicative of a request to generate a claim to be submitted to an insurer on behalf of the healthcare professional and the healthcare facility;
causing display of a form on a network-accessible interface through which an analyst is able to generate the claim;
enabling the analyst to populate the form with information regarding the healthcare professional or the healthcare facility;
receiving second input indicative of a confirmation that the claim is complete;
in response to receiving the second input,
compiling a data structure that is representative of the claim and includes at least some of the information populated into the form; and
transmitting the data structure to the insurer from which reimbursement is sought.

9. The non-transitory medium of claim 8, wherein said examining involves an automated analysis of content of the first and second documents.

10. The non-transitory medium of claim 8, wherein the operations further comprise:
causing transmission of a notification to the healthcare professional so as to indicate that the claim has been submitted to the insurer.

11. The non-transitory medium of claim 10, wherein the notification is transmitted to the first email address associated with the healthcare professional.

12. The non-transitory medium of claim 8, wherein the operations further comprise:
causing transmission of a notification to the healthcare facility so as to indicate that the claim has been submitted to the insurer.

13. The non-transitory medium of claim 12, wherein the notification is transmitted to the second email address associated with the healthcare facility.

14. The non-transitory medium of claim 8, wherein the operations further comprise:
in response to receiving the second input,
employing an artificial intelligence algorithm to autonomously review the information included in the data structure so as to ensure that the data structure complies with guidelines of an insurance plan through which the reimbursement is sought.

15. The non-transitory medium of claim 8, wherein the operations further comprise:
performing a text recognition operation on the first and second documents so as to convert content into machine-encoded text;
creating, for the healthcare professional, a first data structure that includes information derived from the machine-encoded text corresponding to the first document; and
creating, for the healthcare facility, a second data structure that includes information derived from the machine-encoded text corresponding to the second document.

16. The non-transitory medium of claim 15, wherein the operations further comprise:
populating at least one field included in the form with information included in the first data structure or the second data structure.

17. A method comprising:
generating a network-accessible interface that is accessible to an analyst via a computing device;
displaying, on the network-accessible interface, an email received from a healthcare provider regarding a service provided to a patient;
receiving first input indicative of a query for a record associated with the patient;
searching a database of records associated with different patients so as to establish whether a matching record exists;
determining that no record exists for the patient in the database;
displaying, on the network-accessible interface, a form with fields in which the analyst is able to input information regarding the patient;
populating at least one field included in the form with information obtained from the email;
receiving second input indicative of a request to create a record for the patient; and
storing, in the database, a data structure that is created based on the information included in the fields of the form.

18. The method of claim 17, wherein the healthcare provider is a professional or a facility.
